(19) United States
(12) Patent Application Publication
(10) Pub. No.: US 2026/0080540 A1
(43) Pub. Date: Mar. 19, 2026

(54) MEDICAL IMAGE PROCESSING DEVICE, MEDICAL IMAGE PROCESSING METHOD, AND PROGRAM

(71) Applicant: FUJIFILM Corporation, Tokyo (JP)

(72) Inventor: Shumpei KAMON, Kanagawa (JP)

(73) Assignee: FUJIFILM Corporation, Tokyo (JP)

(21) Appl. No.: 19/309,567

(22) Filed: Aug. 25, 2025

(30) Foreign Application Priority Data
Sep. 13, 2024 (JP) 2024-159381

(51) Int. Cl. G06T 7/00; A61B 1/00; G06V 10/25; G06V 10/764; G06V 10/82; G16H 40/63

(52) U.S. Cl. CPC G06T 7/0016; A61B 1/000096; A61B 1/00055; G06V 10/25; G06V 10/764; G06V 10/82; G16H 40/63; G06T 2207/10016; G06T 2207/10068; G06T 2207/20084; G06T 2207/30028; G06T 2207/30092; G06T 2207/30096; G06V 2201/03

(57) ABSTRACT

Provided are a medical image processing device, a medical image processing method, and a program that enable a user to correctly recognize a plurality of recognition results. A medical image processing device of the present disclosure includes a processor, in which the processor is configured to acquire a plurality of time-series medical images, perform first recognition processing on a first medical image among the plurality of time-series medical images, perform second recognition processing different from the first recognition processing on the first medical image or a second medical image, which is later in time series than the first medical image, among the plurality of time-series medical images, notify of a first recognition result of the first recognition processing, notify of a second recognition result of the second recognition processing, and change a notification method for the second recognition result according to a notification state of the first recognition result.

### CROSS-REFERENCE TO RELATED APPLICATION

The present application claims priority under 35 U.S.C § 119(a) to Japanese Patent Application No. 2024-159381 filed on Sep. 13, 2024, which is hereby expressly incorporated by reference, in its entirety, into the present application.

### BACKGROUND OF THE INVENTION

### 1. Field of the Invention

The present invention relates to a medical image processing device, a medical image processing method, and a program, and particularly relates to a technique for controlling notification of a recognition result of a medical image.

### 2. Description of the Related Art

An AI-supported function is known that uses artificial intelligence (AI) to detect a lesion from an image captured by a medical image diagnostic apparatus such as an endoscope or an ultrasound diagnostic apparatus.

For example, WO2021/010225A discloses a computer program for causing a computer to execute a process comprising: acquiring an image captured by an endoscope; inputting the image captured by the endoscope to a first recognizer that recognizes a lesion part based on the image and a second recognizer that recognizes the lesion part with higher recognition accuracy than the first recognizer based on the image; acquiring provisional information including a recognition result recognized by the first recognizer; outputting an image including the acquired provisional information; acquiring confirmation information corresponding to provisional information including a recognition result recognized by the second recognizer; and outputting an image including the acquired confirmation information.

WO2020/017212A discloses an endoscope system for examining an inside of a patient's lumen, the endoscope system comprising: an insertion part that is inserted into the lumen; a camera that images the inside of the lumen to acquire an endoscopic image; a region-of-interest detection unit that detects a region of interest from the endoscopic image; a detection result notification unit that notifies of a detection result of the region of interest; an insertion/removal determination unit that determines whether a step of the examination is an insertion step or a removal step; and a notification controller that causes the detection result notification unit to make a notification according to the step determined by the insertion/removal determination unit.

In addition, WO2020/090729A discloses a medical image processing device comprising: a notification controller that controls notification information included in a medical image to either a notification state in which the notification information is notified of by a notification unit and a non-notification state in which the notification information is not notified of, in which the notification controller sets the notification information to the non-notification state in a case where the medical image satisfies a non-notification condition, and sets the notification information to the notification state after a non-notification maintenance time has elapsed since the medical image no longer satisfies the non-notification condition.

### SUMMARY OF THE INVENTION

These support functions need to notify a user of an AI result in real time during the examination, but, in a case where a plurality of functions are installed, timings of a plurality of notifications may overlap. In a case where the timings of the plurality of notifications overlap, the user may miss one of the notifications or become confused, so that the user may not be able to correctly recognize the notification information.

The present invention has been made in view of such circumstances, and an object of the present invention is to provide a medical image processing device, a medical image processing method, and a program that enable a user to correctly recognize a plurality of recognition results.

In order to achieve the above object, according to a first aspect of the present disclosure, there is provided a medical image processing device comprising: a processor, in which the processor is configured to acquire a plurality of time-series medical images, perform first recognition processing on a first medical image among the plurality of time-series medical images, perform second recognition processing different from the first recognition processing on the first medical image or a second medical image, which is later in time series than the first medical image, among the plurality of time-series medical images, notify of a first recognition result of the first recognition processing, notify of a second recognition result of the second recognition processing, and change a notification method for the second recognition result according to a notification state of the first recognition result.

According to a second aspect of the present disclosure, in the medical image processing device according to the first aspect, it is preferable that the processor is configured to notify of at least one of the first recognition result or the second recognition result as image information.

According to a third aspect of the present disclosure, in the medical image processing device according to the first or second aspect, it is preferable that the processor is configured to notify of at least one of the first recognition result or the second recognition result as audio information.

According to a fourth aspect of the present disclosure, in the medical image processing device according to any one of the first to third aspects, it is preferable that the processor is configured to notify of the second recognition result as a plurality of pieces of information including at least image information and audio information, and change the notification method for any one of the plurality of pieces of information according to the notification state.

According to a fifth aspect of the present disclosure, in the medical image processing device according to the fourth aspect, it is preferable that the processor is configured to change the notification method for the audio information according to the notification state.

According to a sixth aspect of the present disclosure, in the medical image processing device according to any one of the second to fifth aspects, it is preferable that the processor is configured to notify of the first recognition result as first image information to a first display unit that displays the medical image, and notify of the second recognition result as second image information to a second display unit that is different from the first display unit.

According to a seventh aspect of the present disclosure, in the medical image processing device according to any one of the first to sixth aspects, it is preferable that the first recognition processing is region-of-interest detection processing of detecting a region of interest included in the first medical image or region-of-interest classification processing of classifying the region of interest included in the first medical image.

According to an eighth aspect of the present disclosure, in the medical image processing device according to the seventh aspect, it is preferable that the processor is configured to notify of the first recognition result continuously for a predetermined time, and notify of the second recognition result after the predetermined time has elapsed.

According to a ninth aspect of the present disclosure, in the medical image processing device according to any one of the first to eighth aspects, it is preferable that the processor is configured to change the notification method depending on whether or not the first recognition result has been notified of.

According to a tenth aspect of the present disclosure, in the medical image processing device according to any one of the first to ninth aspects, it is preferable that the processor is configured to change the notification method depending on whether or not the first recognition result has been notified of within a predetermined time or whether or not the notification state has changed.

According to an eleventh aspect of the present disclosure, in the medical image processing device according to any one of the first to tenth aspects, it is preferable that the second recognition processing is site recognition processing of recognizing an observation site of the first medical image or the second medical image.

According to a twelfth aspect of the present disclosure, in the medical image processing device according to the eleventh aspect, it is preferable that the processor is configured to visually display a part that is captured and a part that is not captured in the plurality of time-series medical images, as the second recognition result.

According to a thirteenth aspect of the present disclosure, in the medical image processing device according to any one of the first to twelfth aspects, it is preferable that the processor is configured to change at least one of a notification time, a notification timing, or a notification level of the second recognition result, as the notification method.

According to a fourteenth aspect of the present disclosure, in the medical image processing device according to the thirteenth aspect, it is preferable that the processor is configured to change the notification timing of the second recognition result to a point later than a notification timing of the first recognition result.

In order to achieve the above object, according to a fifteenth aspect of the present disclosure, there is provided a medical image processing method comprising: via a processor, acquiring a plurality of time-series medical images; performing first recognition processing on a first medical image among the plurality of time-series medical images; performing second recognition processing different from the first recognition processing on the first medical image or a second medical image, which is later in time series than the first medical image, among the plurality of time-series medical images; notifying of a first recognition result of the first recognition processing; notifying of a second recognition result of the second recognition processing; and changing a notification method for the second recognition result according to a notification state of the first recognition result.

In order to achieve the above object, according to a sixteenth aspect of the present disclosure, there is provided a program for causing a computer to execute the medical image processing method according to the fifteenth aspect. The present disclosure also includes a non-transitory computer-readable storage medium in which the program according to the sixteenth aspect is stored.

In the medical image processing method according to the fifteenth aspect and the program according to the sixteenth aspect, a configuration including the same specific aspects as the medical image processing device described above can be adopted.

According to the present invention, the user can correctly recognize a plurality of recognition results.

### BRIEF DESCRIPTION OF THE DRAWINGS

FIG. 1 is a schematic diagram showing an overall configuration of an endoscope system.

FIG. 2 is a block diagram showing an example of an electric configuration of a medical image processing device.

FIG. 3 is a flowchart showing a medical image processing method.

FIG. 4 is a table showing an example of output timings of a lesion detection result and an observation site recognition result.

FIG. 5 is a diagram showing a display example of a display device.

FIG. 6 is a block diagram showing an example of an electric configuration of a medical image processing device.

FIG. 7 is a flowchart showing a medical image processing method.

FIG. 8 is a flowchart showing a medical image processing method.

### DESCRIPTION OF THE PREFERRED EMBODIMENTS

Hereinafter, preferred embodiments of the present invention will be described with reference to the accompanying drawings. The same components are denoted by the same reference numerals, and overlapping description will not be repeated.

### Overall Configuration of Endoscope System

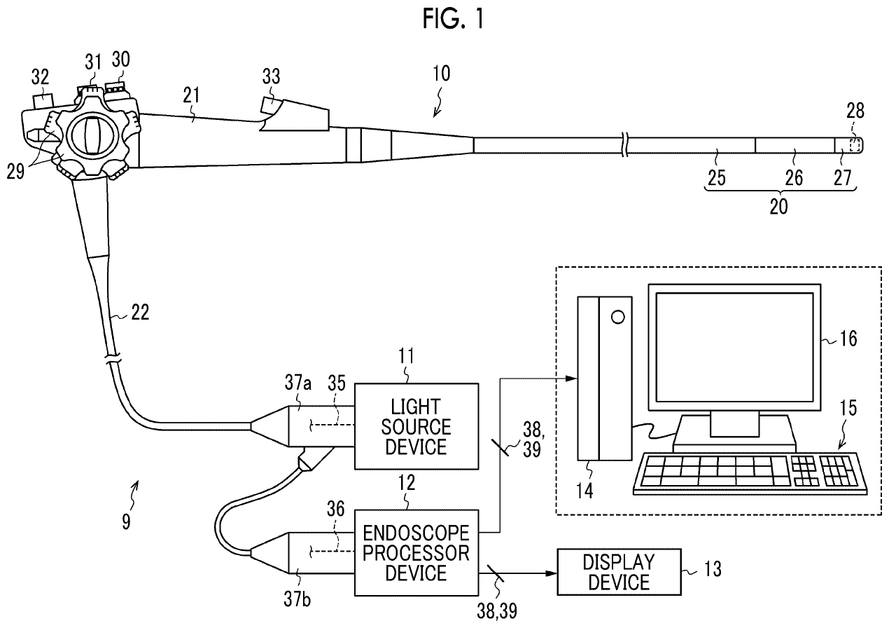

FIG. 1 is a schematic diagram showing an overall configuration of an endoscope system 9 including a medical image processing device according to the present disclosure. As shown in FIG. 1, the endoscope system 9 comprises an endoscope 10, which is an electronic endoscope, a light source device 11, an endoscope processor device 12, a display device 13, a medical image processing device 14, an input device 15, and a display device 16.

The endoscope 10 is used for capturing a plurality of time-series endoscopic images, and is, for example, a flexible endoscope. The endoscope 10 includes an insertion part 20 that is inserted into a subject and that has a distal end and a base end, a hand operation part 21 that is consecutively installed on a base end side of the insertion part 20 and that is held by a user (doctor) for performing various operations, and a universal cord 22 that is consecutively installed with the hand operation part 21.

The insertion part 20 is formed in an elongated shape with a small diameter as a whole. The insertion part 20 is configured by consecutively installing, in order from the base end side to a distal end side, a flexible portion 25 that has flexibility, a bendable portion 26 that can be bent by operating the hand operation part 21, and a distal end portion 27 that incorporates an imaging optical system (objective lens) (not shown), an imaging element 28, and the like.

The imaging element 28 is an imaging element of a complementary metal oxide semiconductor (CMOS) type or a charge coupled device (CCD) type. Image light of an observation site is incident on an imaging surface of the imaging element 28 via an observation window (not shown) that is open to a distal end surface of the distal end portion 27, and an objective lens (not shown) disposed behind the observation window. The imaging element 28 outputs an imaging signal by imaging (converting into an electric signal) the image light of the observation site incident on the imaging surface.

The hand operation part 21 is provided with various operation members that are operated by the user. Specifically, the hand operation part 21 is provided with two types of bending operation knobs 29 that are used for a bending operation of the bendable portion 26, an air/water supply button 30 for an air/water supply operation, and a suction button 31 for a suction operation. In addition, the hand operation part 21 is provided with a still image capturing instruction unit 32 for issuing an instruction to capture a still image 39 of the observation site and a treatment tool inlet port 33 through which a treatment tool (not shown) is inserted into a treatment tool insertion passage (not shown) passing through the insertion part 20.

The universal cord 22 is a connection cord for connecting the endoscope 10 to the light source device 11. The universal cord 22 encompasses a light guide 35, a signal cable 36, and a fluid tube (not shown) which are inserted into the insertion part 20. In addition, a connector 37a that is connected to the light source device 11, and a connector 37b that branches from the connector 37a and that is connected to the endoscope processor device 12 are disposed in an end part of the universal cord 22.

By connecting the connector 37a to the light source device 11, the light guide 35 and the fluid tube are inserted into the light source device 11. Accordingly, necessary illumination light, water, and gas are supplied from the light source device 11 to the endoscope 10 via the light guide 35 and the fluid tube. As a result, illumination light is emitted from an illumination window (not shown) on the distal end surface of the distal end portion 27 toward the observation site. In addition, gas or water is jetted from an air/water supply nozzle (not shown) on the distal end surface of the distal end portion 27 toward an observation window (not shown) on the distal end surface according to a pressing operation of the air/water supply button 30 described above.

By connecting the connector 37b to the endoscope processor device 12, the signal cable 36 and the endoscope processor device 12 are electrically connected to each other. Accordingly, via the signal cable 36, the imaging signal of the observation site is output from the imaging element 28 of the endoscope 10 to the endoscope processor device 12, and a control signal is output from the endoscope processor device 12 to the endoscope 10.

The light source device 11 supplies the illumination light to the light guide 35 of the endoscope 10 via the connector 37a. As the illumination light, light of various wavelength ranges corresponding to an observation purpose, such as white light (light in a white wavelength range or light in a plurality of wavelength ranges), light in one or a plurality of specific wavelength ranges, or a combination thereof is selected. Note that the specific wavelength range is a range narrower than the white wavelength range.

A first example of the specific wavelength range is, for example, a blue or green range of visible range. The wavelength range of the first example includes a wavelength range of greater than or equal to 390 nm and less than or equal to 450 nm or greater than or equal to 530 nm and less than or equal to 550 nm. Light of the first example has a peak wavelength in the wavelength range of greater than or equal to 390 nm and less than or equal to 450 nm or greater than or equal to 530 nm and less than or equal to 550 nm.

A second example of the specific wavelength range is, for example, a red range of visible range. The wavelength range of the second example includes a wavelength range of greater than or equal to 585 nm and less than or equal to 615 nm or greater than or equal to 610 nm and less than or equal to 730 nm. Light of the second example has a peak wavelength in the wavelength range of greater than or equal to 585 nm and less than or equal to 615 nm or greater than or equal to 610 nm and less than or equal to 730 nm.

A third example of the specific wavelength range includes a wavelength range of which a light absorption coefficient varies between oxygenated hemoglobin and reduced hemoglobin. Light of the third example has a peak wavelength in the wavelength range of which the light absorption coefficient varies between the oxygenated hemoglobin and the reduced hemoglobin. The wavelength range of the third example includes a wavelength range of 400±10 nm, 440±10 nm, 470±10 nm, or greater than or equal to 600 nm and less than or equal to 750 nm. Light of the third example has a peak wavelength in the wavelength range of 400±10 nm, 440±10 nm, 470±10 nm, or greater than or equal to 600 nm and less than or equal to 750 nm.

A fourth example of the specific wavelength range is a wavelength range (390 nm to 470 nm) of excitation light that is used for observing (fluorescence observation) fluorescence emitted by a fluorescent substance in a living body and that excites the fluorescent substance.

A fifth example of the specific wavelength range is a wavelength range of infrared light. The wavelength range of the fifth example includes a wavelength range of greater than or equal to 790 nm and less than or equal to 820 nm or greater than or equal to 905 nm and less than or equal to 970 nm. Light of the fifth example has a peak wavelength in the wavelength range of greater than or equal to 790 nm and less than or equal to 820 nm or greater than or equal to 905 nm and less than or equal to 970 nm.

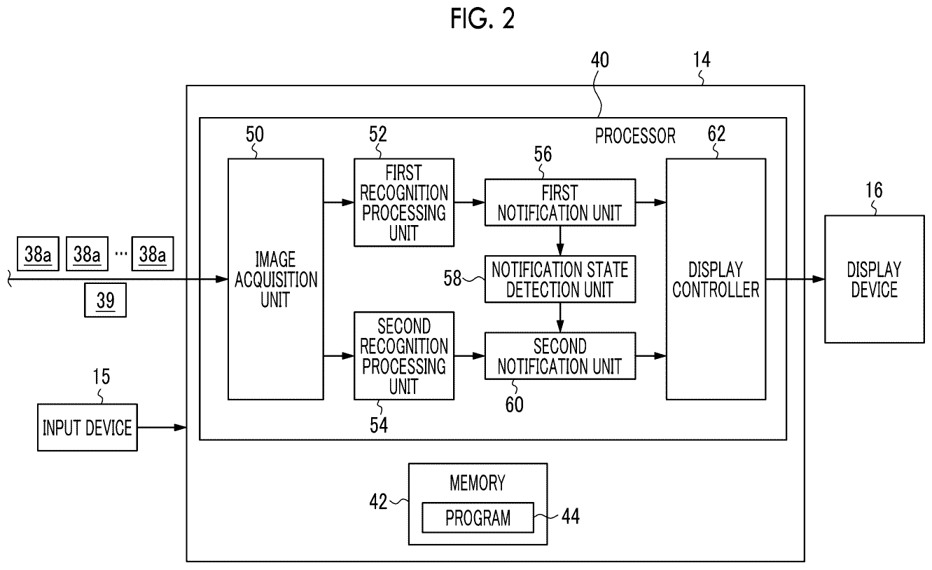

The endoscope processor device 12 controls the operation of the endoscope 10 via the connector 37b and the signal cable 36. In addition, the endoscope processor device 12 generates a time-series video 38 consisting of time-series frame images 38a (see FIG. 2) based on the imaging signal acquired from the imaging element 28 of the endoscope 10 via the connector 37b and the signal cable 36. A frame rate of the video 38 is, for example, 30 frames per second (fps).

Further, in a case where the still image capturing instruction unit 32 is operated by the hand operation part 21 of the endoscope 10, the endoscope processor device 12 acquires one frame image 38a of the video 38 in accordance with a timing of imaging instruction, in parallel with the generation of the video 38, and sets the frame image as the still image 39. Then, the endoscope processor device 12 outputs the generated video 38 and the still image 39 to each of the display device 13 and the medical image processing device 14.

The endoscope processor device 12 may generate (acquire) a special light image having information on the specific wavelength range based on a normal light image obtained by the white light. In this case, the endoscope processor device 12 functions as a special light image acquisition unit. Then, the endoscope processor device 12 obtains a signal of the specific wavelength range by performing calculation based on RGB color information of red, green, and blue or CMY color information of cyan, magenta, and yellow, which is contained in the normal light image.

In addition, the endoscope processor device 12 may generate a feature amount image such as a known oxygen saturation image, for example, based on at least one of the normal light image obtained by the white light or the special light image obtained by the light (special light) of the specific wavelength range. In this case, the endoscope processor device 12 functions as a feature amount image generation unit. The video 38 or the still image 39, each of which includes any of the image of the inside of the living body, the normal light image, the special light image, or the feature amount image, is an example of a medical image obtained by visualizing a result of imaging or measuring a human body for diagnosis and examination purposes based on images. The video 38 is an example of a plurality of time-series medical images.

The display device 13 is connected to the endoscope processor device 12 and displays the video 38 and the still image 39 input from the endoscope processor device 12. The user performs a forward and backward operation of the insertion part 20 while checking the video 38 displayed on the display device 13, and, in a case where a lesion or the like is found in the observation site, the user operates the still image capturing instruction unit 32 to capture a still image of the observation site, and performs diagnosis, biopsy, and the like.

The medical image processing device 14 functions as a diagnosis support device. The medical image processing device 14 notifies the user of information included in the medical image. The information included in the medical image is, for example, a result of recognition processing on the medical image. As the medical image processing device 14, for example, a personal computer comprising a processor is used.

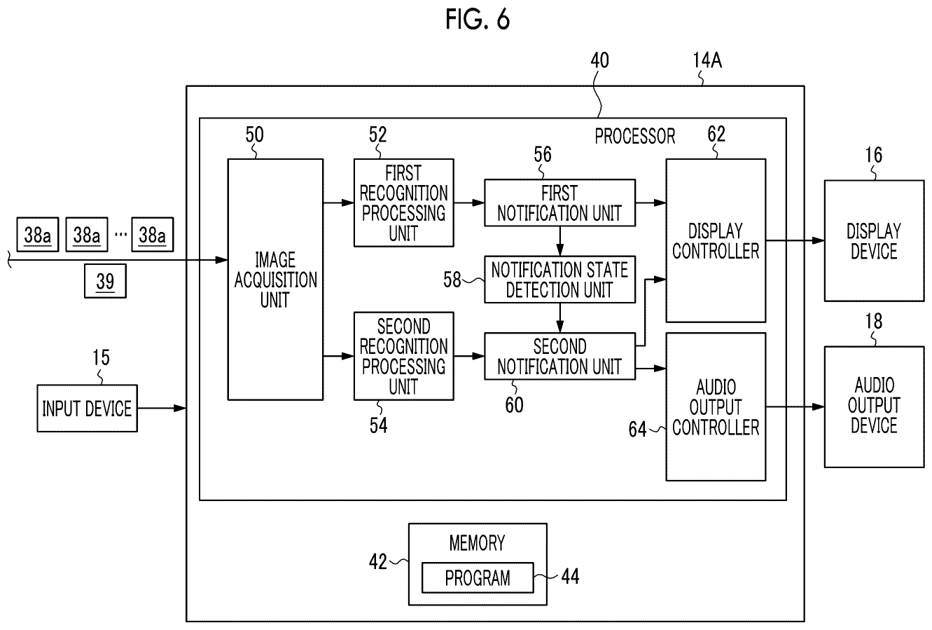

As the input device 15, a keyboard, a mouse, and the like that are connected to a personal computer in a wired or wireless manner are used. As the display device 16, various monitors such as a liquid crystal monitor that can be connected to a personal computer are used. An audio output device 18 (see FIG. 6) may be connected to the medical image processing device 14. A buzzer or a speaker may be used as the audio output device 18.

### First Embodiment

### Configuration of Medical Image Processing Device

FIG. 2 is a block diagram showing an example of an electric configuration of the medical image processing device 14. The medical image processing device 14 shown in FIG. 2 includes a processor 40 and a memory 42. The processor 40 is configured of an arithmetic device such as a central processing unit (CPU). The memory 42 is configured of a storage element such as a random access memory (RAM) and a read only memory (ROM). The memory 42 stores a program 44. The program 44 includes instructions executed by the processor 40. The program 44 may be provided by a computer-readable non-transitory storage medium. In this case, the processor 40 may read the program 44 from the non-transitory storage medium and store the program 44 in the memory 42.

The processor 40 includes an image acquisition unit 50, a first recognition processing unit 52, a second recognition processing unit 54, a first notification unit 56, a notification state detection unit 58, a second notification unit 60, and a display controller 62. Each function of the image acquisition unit 50, the first recognition processing unit 52, the second recognition processing unit 54, the first notification unit 56, the notification state detection unit 58, the second notification unit 60, and the display controller 62 is realized by the processor 40 executing the program 44.

The image acquisition unit 50 sequentially acquires endoscopic images captured by the endoscope 10. Here, the image acquisition unit 50 acquires the video 38 consisting of the time-series frame images 38a from the endoscope processor device 12 using an image input/output interface (not shown) connected to the endoscope processor device 12 (see FIG. 1) in a wired or wireless manner. In addition, in a case where the still image 39 is captured in the middle of imaging the video 38 in the endoscope 10, the image acquisition unit 50 acquires the video 38 and the still image 39 from the endoscope processor device 12.

The image acquisition unit 50 may acquire the video 38 via a non-transitory storage medium, such as a memory card or a hard disk device, instead of directly acquiring the video 38 from the endoscope processor device 12. In addition, the image acquisition unit 50 may acquire the video 38 uploaded to a server, a database, or the like on the Internet via the Internet.

The first recognition processing unit 52 performs first recognition processing on the endoscopic image acquired by the image acquisition unit 50 to acquire a first recognition result. The first recognition processing is, for example, region-of-interest detection processing of detecting a region of interest included in the endoscopic image. The first recognition processing unit 52 includes a convolutional neural network (CNN) that calculates a feature amount of the endoscopic image and that performs recognition processing on a region of interest in the endoscopic image.

The region of interest may include a lesion. The region of interest may include at least one of polyps, cancers, colonic diverticulum, inflammation, endoscopic mucosal resection (EMR) scars or endoscopic submucosal dissection (ESD) scars, clip locations, bleeding points, perforations, vascular atypia, or a treatment tool.

The first recognition processing unit 52 may perform region-of-interest classification processing such as category classification (discrimination) of which category the detected region of interest belongs to among a plurality of categories related to lesions such as “neoplastic”, “non-neoplastic”, and “others”.

The first recognition processing unit 52 is not limited to detecting the region of interest using the CNN, and may analyze a feature amount, such as color, pixel value gradient, shape, and size in the image, through image processing to detect the region of interest.

The second recognition processing unit 54 performs second recognition processing different from first recognition processing on the endoscopic image acquired by the image acquisition unit 50 to acquire a second recognition result. The second recognition processing is, for example, observation site recognition processing of recognizing the observation site of the endoscopic image. The second recognition processing unit 54 includes a CNN that calculates a feature amount of the endoscopic image and that performs observation site recognition processing on the endoscopic image. The second recognition processing unit 54 may include processing of determining whether or not the endoscopic image acquired by the image acquisition unit 50 is an endoscopic image that satisfies predetermined conditions such as the amount of blur, composition, and region.

In a case where the endoscope 10 is a lower endoscope that is inserted from the anus of the subject and that is used to observe the rectum, the large intestine, and the like, the observation site may include at least one of the rectum, the sigmoid colon, the descending colon, the transverse colon, the ascending colon, the cecum, the ileum, or the jejunum. In a case where the endoscope 10 is an upper endoscope that is inserted from the mouth or the nose of the subject and that is used to observe the esophagus, the stomach, and the like, the observation site may include at least one of the pharynx, the esophagus, the stomach, or the duodenum.

The second recognition processing unit 54 is not limited to detecting the region of interest using the CNN, and may analyze a feature amount, such as color, pixel value gradient, shape, and size in the image, through image processing to recognize the observation site.

The first notification unit 56 notifies of the first recognition result of the first recognition processing unit 52. Here, the first notification unit 56 notifies of the first recognition result as image information by displaying the first recognition result on the display device 16 via the display controller 62.

The notification state detection unit 58 detects a notification state of the first notification unit 56. The notification state of the first notification unit 56 includes, for example, whether or not the first recognition result has been notified of.

The second notification unit 60 notifies of the second recognition result of the second recognition processing unit 54. Here, the second notification unit 60 notifies of the second recognition result as image information by displaying the second recognition result on the display device 16 via the display controller 62.

In addition, the second notification unit 60 changes a notification method for the second recognition result depending on a detection result of the notification state detection unit 58, that is, depending on the notification state of the first recognition result. For example, the second notification unit 60 changes at least one of a notification time, a notification timing, or a notification level of the second recognition result, as the notification method.

The display controller 62 controls the display of the display device 16 based on signals from the first notification unit 56 and the second notification unit 60.

As described above, the medical image processing device 14 notifies of at least one of the first recognition result or the second recognition result to the display device 16, as image information. The user can recognize the image information by visually recognizing a screen display of the display device 16.

### Medical Image Processing Method

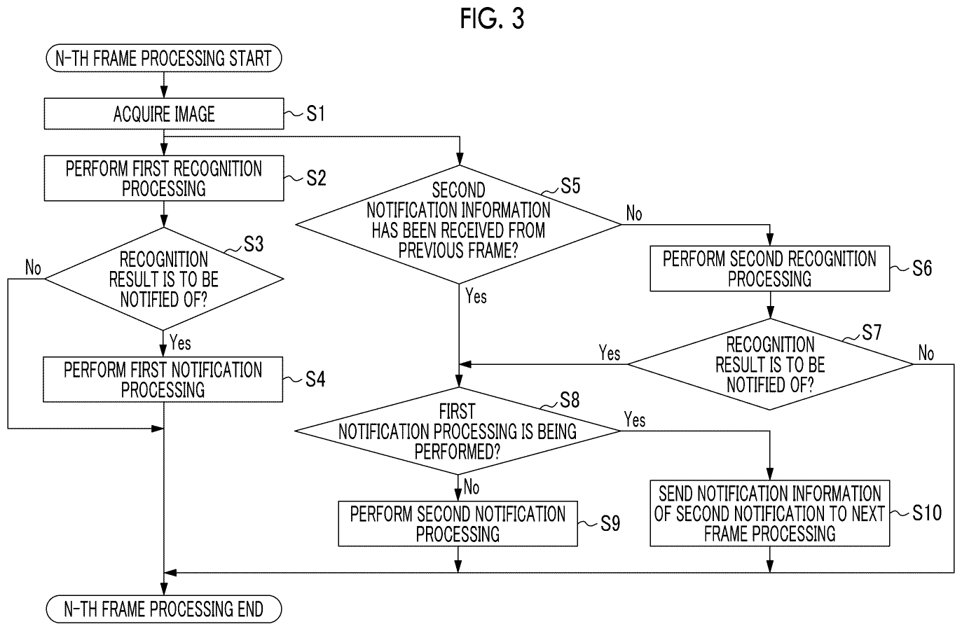

FIG. 3 is a flowchart showing a medical image processing method using the medical image processing device 14. The medical image processing method is realized by the processor 40 executing the program 44. Here, processing of the frame image 38a of an N-th frame of the video 38 will be described.

In step S1, the image acquisition unit 50 acquires the frame image 38a of the N-th frame of the video 38. The frame image 38a acquired in step S1 may be displayed on the display device 16.

In step S2, the first recognition processing unit 52 performs the region-of-interest detection processing, which is the first recognition processing, on the frame image 38a acquired in step S1 to acquire a region-of-interest detection result, which is the first recognition result.

In step S3, the first recognition processing unit 52 determines whether or not to notify of the region-of-interest detection result acquired in step S2. For example, the first recognition processing unit 52 determines to notify of the region-of-interest detection result in a case where the region of interest is detected, and determines not to notify of the region-of-interest detection result in a case where the region of interest is not detected.

In a case where the determination result of step S3 is Yes, that is, in a case where the region-of-interest detection result is to be notified of, the first notification unit 56 performs first notification processing in step S4. The first notification processing is processing of displaying the region-of-interest detection result as image information on the display device 16 via the display controller 62.

On the other hand, in a case where the determination result of step S3 is No, that is, in a case where the region-of-interest detection result is not to be notified of, the first notification unit 56 does not perform the first notification processing.

The processes of steps S5 to S10 are performed in parallel with the processes of steps S2 to S4. The processes of steps S5 to S10 may be performed after the processes of steps S2 to S4 are ended. In step S5, the notification state detection unit 58 determines whether or not second notification information has been received from processing of an (N−1)-th frame, which is a frame preceding the N-th frame. The second notification information includes, for example, an observation site recognition result of the observation site recognition processing on the frame image 38a of the (N−1)-th frame or an earlier frame.

In a case where the determination result of step S5 is No, that is, in a case where the second notification information has not been received, in step S6, the second recognition processing unit 54 performs observation site recognition processing, which is the second recognition processing, on the frame image 38a acquired in step S1, to acquire an observation site recognition result, which is the second recognition result.

In step S7, the second recognition processing unit 54 determines whether or not to notify of the observation site recognition result acquired in step S6. For example, the second recognition processing unit 54 determines to notify of the observation site recognition result in a case where the observation site has been recognized, and determines not to notify of the observation site recognition result in a case where the observation site has not been recognized.

In a case where the determination result of step S7 is No, that is, in a case where the observation site recognition result is not to be notified of, the second notification unit 60 does not perform the second notification processing.

On the other hand, in a case where the determination result of step S7 is Yes, that is, in a case where the observation site recognition result is to be notified of, in step S8, the notification state detection unit 58 determines whether or not the first notification unit 56 is performing the first notification processing. In addition, in a case where the determination result of step S5 is Yes, that is, in a case where the second notification information has been received, similarly, in step S8, the notification state detection unit 58 determines whether or not the first notification unit 56 is performing the first notification processing.

In a case where the determination result in step S8 is No, that is, in a case where the first notification unit 56 is not performing the first notification processing, the second notification unit 60 performs the second notification processing in step S9.

In a case where it is determined in step S5 that the second notification information has not been received, the second notification processing is processing of displaying the observation site recognition result acquired in step S6 as image information on the display device 16 via the display controller 62. In addition, in a case where it is determined in step S5 that the second notification information has been received, the second notification processing is processing of displaying the observation site recognition result as the image information on the display device 16 based on the second notification information of the (N−1)-th frame.

On the other hand, in a case where the determination result in step S8 is Yes, that is, in a case where the first notification unit 56 is performing the first notification processing, in step S10, the second notification unit 60 does not perform the second notification processing and sends the second notification information to processing of the next frame, that is, an (N+1)-th frame.

In a case where it is determined in step S5 that the second notification information has not been received, the second notification information to be sent to the processing of the (N+1)-th frame is the second notification information of the N-th frame, and includes the observation site recognition result acquired in step S6. In a case where it is determined in step S5 that the second notification information has been received, the second notification information is the second notification information received from the processing of the (N−1)-th frame, which is the previous frame.

As described above, according to the medical image processing method, in a case where the first notification processing is performed, a notification timing of the second notification processing is changed to a point later than a notification timing of the first notification processing. By performing this processing for each frame, it is possible to prevent the notification timing of the first notification processing and the notification timing of the second notification processing from overlapping. This prevents the user from missing one of the recognition results, and allows the user to correctly recognize a plurality of recognition results. The change in notification timing is an example of “changing a notification method for the second recognition result” in the present disclosure.

Here, although the notification timing of the second notification processing is changed in a case where the first notification processing is performed, the notification method may be changed depending on the presence or absence of the notification of the first notification processing in the temporal vicinity or depending on the presence or absence of the change in notification state of the first notification processing. The temporal vicinity may refer to a time period within a predetermined time preceding the present time.

For example, the notification timing of the second notification processing may be changed in a case where the first notification processing is performed within a predetermined time preceding the present time. In addition, the notification timing of the second notification processing may be changed in a case where there is a change in notification state of the first notification processing within a predetermined time preceding the present time.

In addition, the first notification unit 56 may perform the first notification processing continuously for a predetermined time. The second notification unit 60 may perform the second notification processing after the predetermined time has elapsed.

For example, in step S3, the first recognition processing unit 52 may determine to notify of the region-of-interest detection result in a case where a predetermined time has not elapsed since the last detection of the region of interest, and may determine not to notify of the region-of-interest detection result in a case where the predetermined time has elapsed since the last detection of the region of interest.

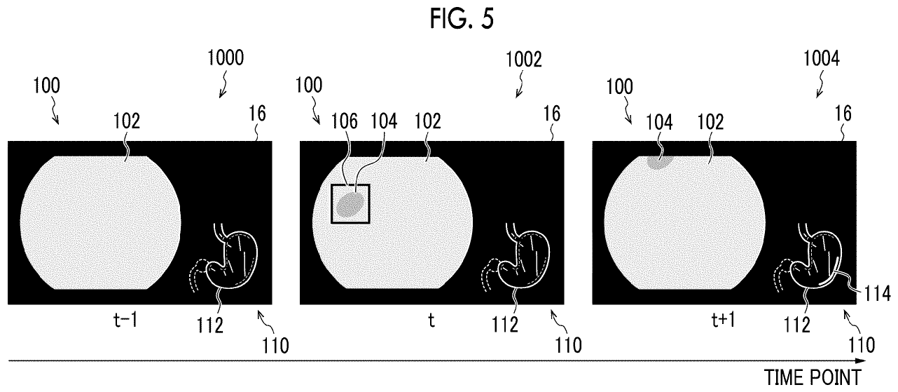

The first notification processing may be, for example, processing of displaying a closed rectangular frame surrounding the detected region of interest. Lines of the frame do not have to be closed. In addition, the first notification processing may be assist circle processing of illuminating an assist circle, which is closer to the detected region of interest, in a predetermined color among an arc-shaped assist circle displayed along a left edge of a medical image (see an endoscopic image 102 shown in FIG. 5) and an arc-shaped assist circle displayed along a right edge of the medical image. The first notification processing may be processing in which the processing of displaying the rectangular frame and the assist circle processing are combined.

In addition, in order not to interfere with the user's observation of the medical image, the first notification unit 56 keeps the assist circle illuminated continuously for a predetermined time (for example, within one second) after detecting a lesion, as the assist circle processing. In a case where the second recognition processing unit 54 performs the second recognition processing on the medical image captured while the assist circle is illuminated after the detection of the lesion, the second notification unit 60 may perform the second notification processing after a predetermined time has elapsed (after the assist circle is turned off).

### Screen Display Example

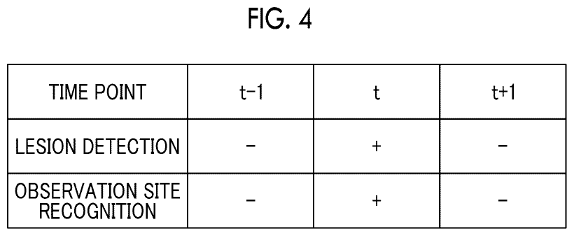

FIG. 4 is a table showing an example of an output timing of the lesion detection of the first recognition processing unit 52 and an output timing of the observation site recognition of the second recognition processing unit 54. Here, the lesion is detected at time point t, and the lesion is not detected at time point (t−1) before time point t and time point (t+1) after time point t. Similarly, the observation site is recognized at time point t, and the observation site is not recognized at time point (t−1) before time point t and at time point (t+1) after time point t.

FIG. 5 is a diagram showing a display example of the display device 16 for the example of the timing shown in FIG. 4. In FIG. 5, 1000 indicates a screen of the display device 16 at time point (t−1), 1002 indicates a screen of the display device 16 at time point t, and 1004 indicates a screen of the display device 16 at time point (t+1).

As shown in 1000 of FIG. 5, at time point (t−1), on the display device 16, an endoscopic image 102 is displayed on a first display unit 100, which is a left region in FIG. 5, and a schematic diagram 112 is displayed on a second display unit 110, which is a right region in FIG. 5. At time point (t−1), the lesion detection result and the observation site recognition result are not displayed.

As shown in 1002 of FIG. 5, at time point t, on the display device 16, the endoscopic image 102 is displayed on the first display unit 100 and the schematic diagram 112 is displayed on the second display unit 110. In addition, on the display device 16, a frame 106 (an example of “first image information”) surrounding a lesion 104 (an example of a “first recognition result”), which is the lesion detection result, is superimposed and displayed on the endoscopic image 102. Meanwhile, at time point t, the observation site recognition result is not displayed.

As shown in 1004 of FIG. 5, at time point (t+1), on the display device 16, the endoscopic image 102 is displayed on the first display unit 100 and the schematic diagram 112 is displayed on the second display unit 110. In addition, on the display device 16, an observation position mark 114 (an example of “second image information”) is superimposed and displayed on the schematic diagram 112 at a position of an observation site (an example of a “second recognition result”), which is the observation site recognition result of the schematic diagram 112. A part that has already been imaged in the video 38 and a part that has not yet been imaged are visually displayed by the observation position mark 114. In addition, the lesion detection result is not displayed at time point (t+1).

As shown in FIG. 4, the lesion is detected at time point t, and the observation site is recognized, but, in a case where both recognition results of the lesion detection result and the observation site recognition result are displayed on the display device 16 at time point t, there is a risk that the user may miss one of the recognition results. Therefore, as shown in FIG. 5, the observation site recognition result is displayed at time point (t+1) at which the display of the lesion detection result has ended, thereby avoiding a situation where the recognition results are displayed simultaneously. In this way, in a case where the lesion detection and the observation site recognition are performed, priority is given to lesion detection that is highly likely to be performed in real time.

Here, in FIG. 5, the left region of the display device 16 is the first display unit 100, and the right region is the second display unit 110, but the arrangement of the first display unit 100 and the second display unit 110 is not limited to this example. In addition, one of the display device 16 and a display device different from the display device 16 may be used as the first display unit 100 and the other may be used as the second display unit 110.

### Second Embodiment

### Configuration of Medical Image Processing Device

FIG. 6 is a block diagram showing an example of an electric configuration of a medical image processing device 14A. The medical image processing device 14A shown in FIG. 6 includes an audio output controller 64. The audio output controller 64 controls the audio output of the audio output device 18 of the endoscope system 9 based on the signal from the second notification unit 60.

The second notification unit 60 notifies of the second recognition result as image information by displaying the second recognition result on the display device 16 via the display controller 62. Further, the second notification unit 60 notifies of the second recognition result as audio information by causing the audio output device 18 to output the second recognition result by audio via the audio output controller 64.

As described above, the medical image processing device 14A notifies of the second recognition result as a plurality of pieces of information including at least the image information and the audio information. The medical image processing device 14A may notify of at least one of the first recognition result or the second recognition result as audio information via the audio output device 18. The user can recognize the audio information by listening to the audio output from the audio output device 18.

### Medical Image Processing Method

The second notification unit 60 may change the notification method for any one of the plurality of pieces of information including the image information and the audio information depending on the detection result of the notification state detection unit 58, that is, depending on the notification state of the first recognition result. The notification method to be changed may be the notification method for the audio information.

### Third Embodiment

### Medical Image Processing Method

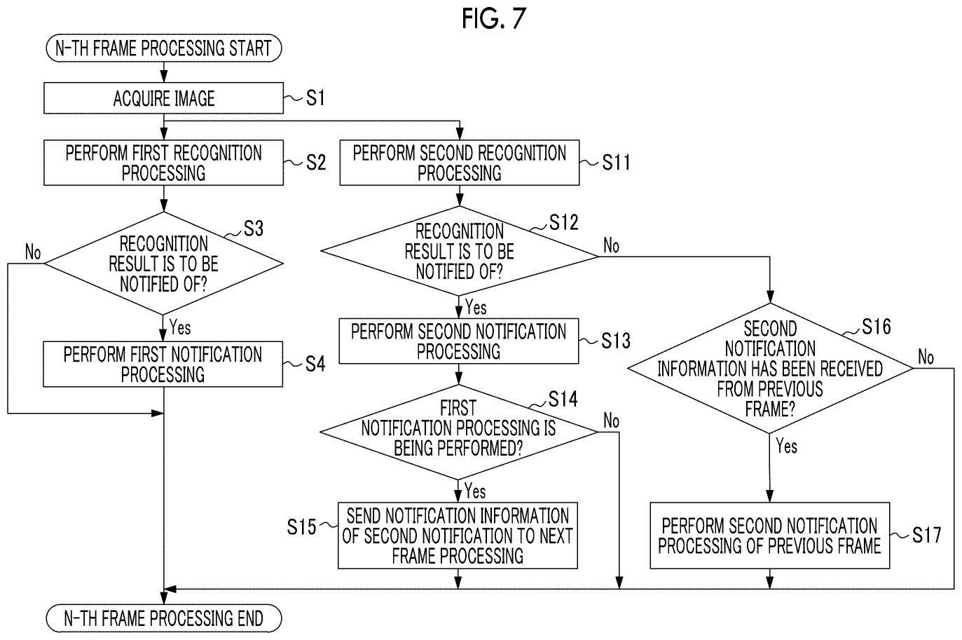

FIG. 7 is a flowchart showing a medical image processing method according to a third embodiment using the medical image processing device 14 or the medical image processing device 14A.

The processes of steps S1 to S4 are the same as those in the medical image processing method according to the first embodiment. In step S11, the second recognition processing unit 54 performs the observation site recognition processing as the second recognition processing on the frame image 38a acquired in step S1 to acquire an observation site recognition result, which is the second recognition result.

In step S12, the second recognition processing unit 54 determines whether or not to notify of the observation site recognition result acquired in step S11.

In a case where the determination result of step S12 is Yes, that is, in a case where the observation site recognition result is to be notified of, the second notification unit 60 performs second notification processing in step S13. The second notification processing is, for example, processing of displaying the observation site recognition result acquired in step S11 as image information on the display device 16 via the display controller 62.

Subsequently, in step S14, the notification state detection unit 58 determines whether or not the first notification unit 56 is performing the first notification processing. In a case where the determination result in step S14 is Yes, that is, in a case where the first notification unit 56 is performing the first notification processing, in step S15, the second notification unit 60 sends the second notification information of the N-th frame to processing of the next frame, that is, the (N+1)-th frame. The second notification information of the N-th frame includes the observation site recognition result acquired in step S11.

On the other hand, in a case where the determination result in step S14 is No, that is, in a case where the first notification unit 56 is not performing the first notification processing, the second notification unit 60 does not perform the process of step S15.

In addition, in a case where the determination result in step S12 is No, that is, in a case where the observation site recognition result is not to be notified of, in step S16, the notification state detection unit 58 determines whether or not the second notification information of the (N−1)-th frame has been received from processing of the (N−1)-th frame, which is a frame preceding the N-th frame.

In a case where the determination result in step S16 is Yes, that is, in a case where the second notification information has been received from the processing of the (N−1)-th frame, the second notification unit 60 performs the second notification processing of the (N−1)-th frame in step S17. The second notification processing is processing of displaying the observation site recognition result as the image information on the display device 16 based on the second notification information received frame the processing of the (N−1)-th frame.

On the other hand, in a case where the determination result in step S16 is No, that is, in a case where the second notification information has not been received, the second notification unit 60 does not perform the process of step S17.

As described above, by extending the notification time of the second notification processing in a case where the first notification processing is being performed, it is possible to prevent the user from missing the second recognition result. The extension of the notification time is an example of “changing a notification method for the second recognition result” in the present disclosure.

### Fourth Embodiment

### Medical Image Processing Method

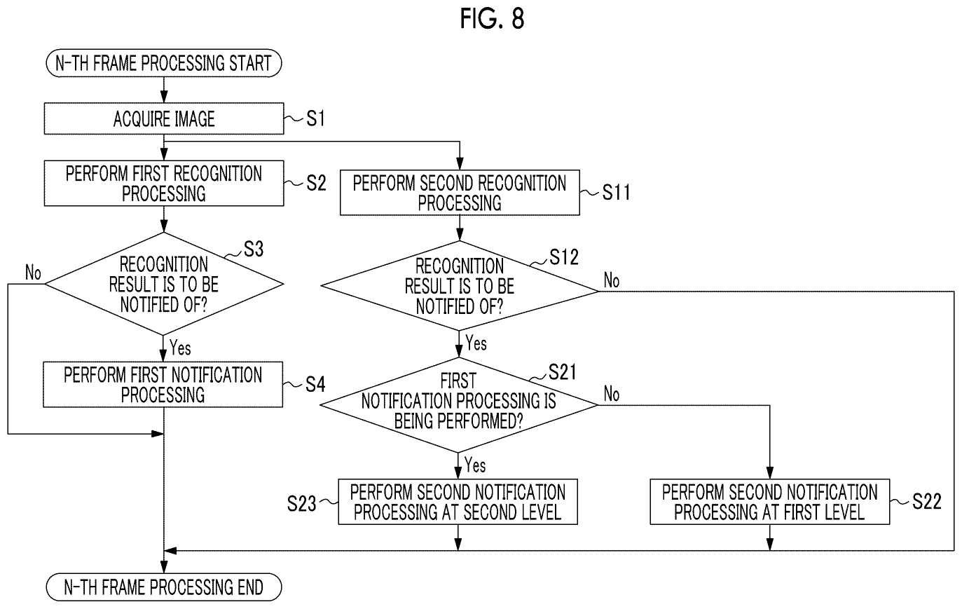

FIG. 8 is a flowchart showing a medical image processing method according to a fourth embodiment using the medical image processing device 14 or the medical image processing device 14A. Here, an example will be described in which the first recognition processing unit 52 performs observation site recognition processing as the first recognition processing, and the second recognition processing unit 54 performs region-of-interest detection processing as the second recognition processing.

The processes of steps S1 to S4 are the same as those in the medical image processing method according to the first embodiment. In step S11, the second recognition processing unit 54 performs the region-of-interest detection processing as the second recognition processing on the frame image 38a acquired in step S1 to acquire a region-of-interest detection result, which is the second recognition result.

In step S12, the second recognition processing unit 54 determines whether or not to notify of the region-of-interest detection result acquired in step S11.

In a case where the determination result of step S12 is No, that is, in a case where the region-of-interest detection result is not be notified of, the second notification unit 60 does not perform any notification.

On the other hand, in a case where the determination result of step S12 is Yes, that is, in a case where the region-of-interest detection result is to be notified of, in step S21, the notification state detection unit 58 determines whether or not the first notification unit 56 is performing the first notification processing.

In a case where the determination result in step S21 is No, that is, in a case where the first notification unit 56 is not performing the first notification processing, the second notification unit 60 performs the second notification processing at a first notification level in step S22. The second notification processing is processing of notifying of the region-of-interest detection result acquired in step S11. The notification level may be a certain degree of emphasis in screen display, a certain audio volume, or the like.

On the other hand, in a case where the determination result in step S21 is Yes, that is, in a case where the first notification unit 56 is performing the first notification processing, the second notification unit 60 performs the second notification processing at a second notification level higher than the first notification level in step S23. For example, a thickness of a frame surrounding the region of interest in the screen display may be thicker than the first notification level, brightness of a color of the frame surrounding the region of interest in the screen display may be higher than the first notification level, and an audio volume for notifying of the region of interest in the audio output may be higher than the first notification level.

As described above, by increasing the notification level of the second notification processing in a case where the first notification processing is being performed, it is possible to prevent the user from missing the second recognition result. The change in notification level is an example of “changing a notification method for the second recognition result” in the present disclosure.

### Modification Example

Although an example in which the first recognition processing and the second recognition processing are performed on the frame image 38a of the N-th frame of the video 38 has been described so far, the first recognition processing may be performed on the frame image 38a of the N-th frame (an example of a “first medical image”), and the second recognition processing may be performed on the frame images 38a (an example of a “second medical image”) from the (N+1)-th frame onwards, which are later in time series than the N-th frame.

The first recognition processing and the second recognition processing may be any of region-of-interest detection processing, observation site recognition processing, lesion recognition processing, or examination state recognition processing. The lesion recognition processing is processing of recognizing information on at least one of a size, a disease type, or a shape of the lesion. The examination state recognition processing is processing of recognizing an examination state such as a treatment state.

In the first to third embodiments, the first recognition processing is main recognition processing, and the second recognition processing is sub-recognition processing. On the other hand, in the fourth embodiment, the second recognition processing is the main recognition processing, and the first recognition processing is the sub-recognition processing. It is preferable that functions such as lesion detection, which have a high risk of being missed or which require faster response speed, are set as the main recognition processing.

Although the endoscopic image has been described as an example of the medical image, the medical image processing device 14 and the medical image processing device 14A can be applied to a medical image such as a capsule endoscopic image and an ultrasound image.

### Configuration of Medical Image Processing Device

In the present embodiment, each processing is executed by any computer. In addition, any computer may execute the processing using a processor, a program, or a combination thereof. Any computer may be a general-purpose computer, a computer for a specific use, a system such as a workstation, or other hardware elements capable of executing a program.

The processor may be configured by one or more pieces of hardware, and the type of hardware is not limited. For example, the processor may be configured by a programmable logic device such as a central processing unit (CPU), a micro processing unit (MPU), or a field programmable gate array (FPGA), a dedicated circuit for executing specific processing such as an application specific integrated circuit (ASIC), or hardware such as a graphics processing unit (GPU) or a neural processing unit (NPU). In addition, the processor has each unit or each means that executes various types of processing in the present embodiment. In addition, the types of hardware may be a combination of different types of hardware. In a case where a plurality of pieces of hardware are configured to execute one or a plurality of processes of a certain processor, the plurality of pieces of hardware may be present in devices physically separated from each other, or may be present in the same device. In addition, in any of the embodiments, the order of each processing executed by the processor is not limited to the above order and may be changed as appropriate. The hardware is configured by an electric circuit (circuitry) in which circuit elements such as semiconductor elements are combined.

Further, the present embodiment may be realized by hardware, software, firmware, microcode, or a combination thereof. Software, firmware, and microcode are configured by a program. In addition, the program may be, for example, a program module group, and each function thereof may be realized by a processor configured to execute each function. The program may be a program code or a plurality of code segments stored in one or a plurality of non-transitory computer-readable media (for example, a storage medium or other storage). The program may be divided and stored in a plurality of non-transitory computer-readable media present in devices physically separate from each other. The program code or the code segment may represent any combination of a procedure, a function, a subprogram, a routine, a subroutine, a module, a software package, a class, an instruction, a data structure, or a program statement. The program code or the code segment may be connected to another code segment or a hardware circuit by transmitting and receiving information, data, an argument, a parameter, or memory contents.

### Other

The technical scope of the present invention is not limited to the scope described in the above embodiments. The configurations and the like in each embodiment can be combined as appropriate among the respective embodiments without departing from the scope of the present invention.

### EXPLANATION OF REFERENCES

9: endoscope system
10: endoscope
11: light source device
12: endoscope processor device
13: display device
14, 14A: medical image processing device
15: input device
16: display device
18: audio output device
20: insertion part
21: hand operation part
22: universal cord
25: flexible portion
26: bendable portion
27: distal end portion
28: imaging element
29: bending operation knob
30: air/water supply button
31: suction button
32: still image capturing instruction unit
33: treatment tool inlet port
35: light guide
36: signal cable
37a: connector
37b: connector
38: video
38a: frame image
39: still image
40: processor
42: memory
44: program
50: image acquisition unit
52: first recognition processing unit
54: second recognition processing unit
56: first notification unit
58: notification state detection unit
60: second notification unit
62: display controller
64: audio output controller
100: first display unit
102: endoscopic image
104: lesion
106: frame
110: second display unit
112: schematic diagram
114: observation position mark
S1 to S10: step of medical image processing method
S11 to S17: step of medical image processing method
S21 to S23: step of medical image processing method

What is claimed is:

1. A medical image processing device comprising:
a processor,
wherein the processor is configured to
acquire a plurality of time-series medical images,
perform first recognition processing on a first medical image among the plurality of time-series medical images,
perform second recognition processing different from the first recognition processing on the first medical image or a second medical image, which is later in time series than the first medical image, among the plurality of time-series medical images,
notify of a first recognition result of the first recognition processing,
notify of a second recognition result of the second recognition processing, and
change a notification method for the second recognition result according to a notification state of the first recognition result.

2. The medical image processing device according to claim 1,
wherein the processor is configured to notify of at least one of the first recognition result or the second recognition result as image information.

3. The medical image processing device according to claim 1,
wherein the processor is configured to notify of at least one of the first recognition result or the second recognition result as audio information.

4. The medical image processing device according to claim 1,
wherein the processor is configured to
notify of the second recognition result as a plurality of pieces of information including at least image information and audio information, and
change the notification method for any one of the plurality of pieces of information according to the notification state.

5. The medical image processing device according to claim 4,
wherein the processor is configured to change the notification method for the audio information according to the notification state.

6. The medical image processing device according to claim 2,
wherein the processor is configured to
notify of the first recognition result as first image information to a first display unit that displays the medical image, and
notify of the second recognition result as second image information to a second display unit that is different from the first display unit.

7. The medical image processing device according to claim 1,
wherein the first recognition processing is region-of-interest detection processing of detecting a region of interest included in the first medical image or region-of-interest classification processing of classifying the region of interest included in the first medical image.

8. The medical image processing device according to claim 7,
wherein the processor is configured to
notify of the first recognition result continuously for a predetermined time, and
notify of the second recognition result after the predetermined time has elapsed.

9. The medical image processing device according to claim 1,
wherein the processor is configured to change the notification method depending on whether or not the first recognition result has been notified of.

10. The medical image processing device according to claim 1,
wherein the processor is configured to change the notification method depending on whether or not the first recognition result has been notified of within a predetermined time or whether or not the notification state has changed.

11. The medical image processing device according to claim 1,
wherein the second recognition processing is site recognition processing of recognizing an observation site of the first medical image or the second medical image.

12. The medical image processing device according to claim 11,
wherein the processor is configured to visually display a part that is captured and a part that is not captured in the plurality of time-series medical images, as the second recognition result.

13. The medical image processing device according to claim 1,
wherein the processor is configured to change at least one of a notification time, a notification timing, or a notification level of the second recognition result, as the notification method.

14. The medical image processing device according to claim 13,
wherein the processor is configured to change the notification timing of the second recognition result to a point later than a notification timing of the first recognition result.

15. A medical image processing method comprising:
via a processor,
acquiring a plurality of time-series medical images;
performing first recognition processing on a first medical image among the plurality of time-series medical images;
performing second recognition processing different from the first recognition processing on the first medical image or a second medical image, which is later in time series than the first medical image, among the plurality of time-series medical images;
notifying of a first recognition result of the first recognition processing;
notifying of a second recognition result of the second recognition processing; and
changing a notification method for the second recognition result according to a notification state of the first recognition result.

16. A non-transitory, computer-readable tangible recording medium which records thereon a program for causing, when read by a computer, the computer to execute the medical image processing method according to claim 15.
